(19) United States
(12) Patent Application Publication
(10) Pub. No.: US 2022/0344062 A1
(43) Pub. Date: Oct. 27, 2022

(54) Software Management System for Guardian Patient/Resident Care

(71) Applicants: Tyrone Jeffrey Nance, Long Beach, CA (US); Emily Elois Woodman-Nance, Long Beach, CA (US)

(72) Inventors: Tyrone Jeffrey Nance, Long Beach, CA (US); Emily Elois Woodman-Nance, Long Beach, CA (US)

(21) Appl. No.: 17/730,902

(22) Filed: Apr. 27, 2022

Related U.S. Application Data
(60) Provisional application No. 63/180,575, filed on Apr. 27, 2021.

(51) Int. Cl. G16H 80/00; G06F 21/32; G16H 10/60

(52) U.S. Cl. CPC G16H 80/00; G06F 21/32; G16H 10/60

(57) ABSTRACT

A system is described herein that provides a guardian of a patient or a resident at a facility with access to an application or an engine executable on a computing device to securely retrieve health or wellness information associated with the patient or the resident at the facility in real-time.

### CROSS-REFERENCE TO RELATED APPLICATIONS SECTION

This application is a U.S. Non-Provisional patent application that claims priority to U.S. Provisional Patent Application Ser. 63/180,575 filed on Apr. 27, 2021, the entire contents of which are hereby incorporated by reference in their entirety.

### FIELD OF THE EMBODIMENTS

The present invention relates to the use of a network environment for facilitating patient/resident health or wellness updates between guardians of the patient/resident and skilled healthcare professionals treating the patient/resident at a healthcare facility.

### BACKGROUND OF THE EMBODIMENTS

As of 2017, more than 15,500 skilled nursing facilities provide care to more than 1.35 million people in the United States who needed assistance with their activities of daily living, including getting out of bed, getting dressed, feeding themselves, and showering, or who had cognitive difficulties, such as from dementia. Skilled nursing facilities also provide post-acute skilled nursing care and rehabilitative services. Additionally, California ranks #2 with nearly 1,198 accredited skilled nursing facilities.

Skilled nursing facility patient care is significant to families and guardians. Currently, there are no forms of communication from nursing facilities to guardians without face-to-face interaction. Healthcare facilities are limited on the type of information for patients/residents that can be released through phone inquiries pursuant to The Health Insurance Portability and Accountability Act (HIPAA). It becomes quite problematic for a guardian of a patient/resident to obtain real-time patient care information over the phone.

Many skilled nursing facilities have incorporated programs to help provide medical staff, doctors, pharmacists, radiologists, and other health care practitioners with relatively updated information. However, this information is only available “in-house” and is only available in person to the guardian or by mail with proper medical authorization release.

Nonetheless, this creates a burden to guardians who seek updated information on their family member's care. Some guardians may live out of state and/or travel from various areas to visit the patient/resident. The lack of accessibility creates an undue burden on the guardian in their attempt to obtain real-time information on their family members. Efficient communication between a guardian and one or more skilled healthcare professionals will improve the availability of patient/resident wellness information and ultimately reduce the amount of time staff at these facilities spend updating patient guardians and relatives directly. For example, communication regarding current and historical values of various biometrics of the patient/resident may prompt a follow-up action with facility staff or provide peace of mind for the patients/residents guardian(s). Another example is seen when communicating regarding the patients/residents diet or activity, which can provide important information that allows the patients/residents guardian to share with other family members who may visit the patient/resident, and hence reducing staff from excessive and redundant questions.

Therefore, a need exists to implement efficient communication between a patients/residents guardian and skilled healthcare workers at healthcare facilities (such as hospitals, nursing facilities, home care facilities, hospice care facilities, rehabilitative facilities, etc.) to improve the availability of patient/resident health and wellness information.

### SUMMARY OF THE EMBODIMENTS

The present invention and its embodiments relate to the use of a network environment for facilitating patient/resident health or wellness updates between guardians of the patient/resident and skilled healthcare professionals treating the patient/resident at a healthcare facility.

An embodiment of the present invention describes a system. The system includes, at least, a first computing device associated with a guardian of a patient or a resident at a facility, a second computing device associated with a healthcare worker of the facility (e.g., a doctor, a surgeon, a nurse, a manager, a counselor, a psychiatrist, a therapist, a psychologist, or a physicians assistant, among others not explicitly listed herein), at least one database, and a network configured to connect the first computing device, the second computing device, and the at least one database. The facility is a hospital, a nursing facility, a home care facility, a home health facility, a hospice care facility, a palliative care facility, an assisted living facility, a senior housing facility, a residential care facility, an adult day care facility, an ambulance service, a hospital, a physical therapy facility, a mental health facility, a behavioral health facility, or an addiction rehabilitative facility, among others not explicitly listed herein.

Moreover, it should be appreciated that the guardian is associated with a first access level associated with the health/wellness care updates and the healthcare worker of the facility is associated with a second access level associated with the health/wellness care updates, where the second access level is greater than the first access level.

Each of the first computing device and the second computing device comprise at least an engine and a graphical user interface (GUI). The at least one database is configured to store a first profile associated with the patient or the resident and a second profile associated with the guardian. The first profile houses medical documentation or records associated with the patient or the resident.

The engine of the first computing device is configured to: receive login credentials via the GUI from the guardian. The login credentials may be a username, a password, or a biometric identification means (e.g., fingerprint identification, face recognition identification, palm print identification, iris recognition, or retina recognition, among others not explicitly listed herein). The engine of the first computing device is further configured to: query the at least one database to compare the login credentials with information stored in the second profile, identify a match between the login credentials and a subset of the information in the second profile, confirm an identity of the guardian, and grant the guardian access to view real-time health or wellness information associated with the patient or the resident. The real-time health or wellness information may include: vitals associated with the patient or the resident (such as blood pressure, pulse rate, body temperature, blood sugar level, and respiration rate, etc.), medications of the patient or the resident, meals eaten by the patient or the resident, activities engaged in by the patient or the resident, doctors' orders or instructions for the patient or the resident, and/or dietary restrictions for the patient or the resident, among others not explicitly listed herein.

Moreover, the engine of the first computing device is further configured to: determine, from the login credentials, if the guardian has a legal authorization to view the health/wellness care updates associated with the patient or the resident. In response to a failure to identify the guardian as having the legal authorization to view the health/wellness care updates associated with the patient or the resident, the engine is further configured to: transmit a notification to a third-party user or the facility to confirm or deny the authorization and prohibit the guardian from viewing the health/wellness care updates associated with the patient or the resident.

Moreover, it should be appreciated that retrieval of the medical documentation or the records from the at least one database is compliant with Health Insurance Portability and Accountability Act (HIPAA) governance. In other embodiments, the engine may include a question and response (Q & R) component that allows the guardian to ask a question and receive an answer from the healthcare worker of the facility. In some embodiments, the engine may include a share component that allows the guardian to share meetings or textual information to a third-party. Further, in other embodiments, the engine further comprises a facility news component that provides the guardian with real-time information regarding the facility.

### BRIEF DESCRIPTION OF THE DRAWINGS

FIG. 1 depicts a block diagram of a system, in accordance with embodiments of the present invention.

FIG. 2A, FIG. 2B, FIG. 2C and FIG. 2D depict schematic diagrams of a patients/residents hub/dashboard displayable on a graphical user interface (GUI) of a computing device, in accordance with embodiments of the present invention.

FIG. 3A, FIG. 3B, and FIG. 3C depict schematic diagrams of a patients/residents vitals displayable on a GUI of a computing device, in accordance with embodiments of the present invention.

FIG. 4A, FIG. 4B, and FIG. 4C depict schematic diagrams of patient/resident care information displayable on a GUI of a computing device, in accordance with embodiments of the present invention.

FIG. 5 depicts a schematic diagram of patient/resident activity information displayable on a GUI of a computing device, in accordance with embodiments of the present invention.

FIG. 6 depicts a schematic diagram of a question and response (Q & R) feature/component of a system displayable on a GUI of a computing device, in accordance with embodiments of the present invention.

FIG. 7 depicts a schematic diagram of a share feature/component of a system displayable on a GUI of a computing device, in accordance with embodiments of the present invention.

FIG. 8 depicts a schematic diagram of a facility news feature/component of a system displayable on a GUI of a computing device, in accordance with embodiments of the present invention.

FIG. 9 is a block diagram of a computing device included within the computer system of FIG. 1, in accordance with embodiments of the present invention.

### DESCRIPTION OF THE PREFERRED EMBODIMENTS

The preferred embodiments of the present invention will now be described with reference to the drawings. Identical elements in the various figures are identified with the same reference numerals.

Reference will now be made in detail to each embodiment of the present invention. Such embodiments are provided by way of explanation of the present invention, which is not intended to be limited thereto. In fact, those of ordinary skill in the art may appreciate upon reading the present specification and viewing the present drawings that various modifications and variations can be made thereto.

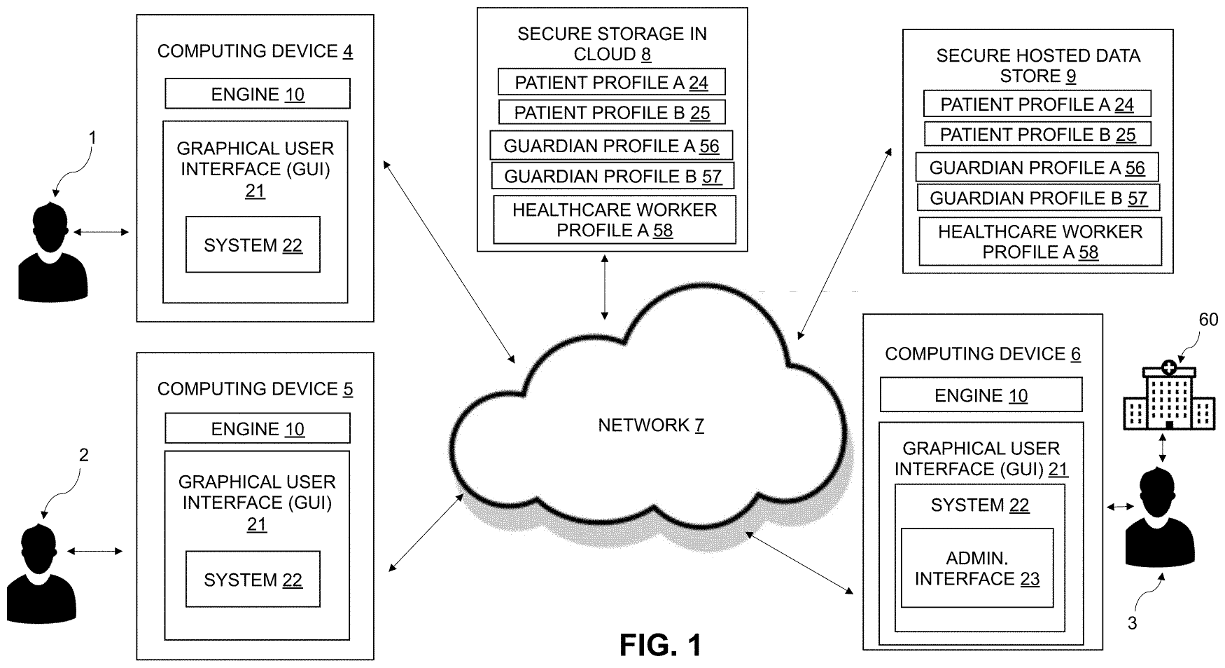

The present invention describes a system, as shown in FIG. 1. In general, the system of FIG. 1 provides a patient/resident guardian access to a software management system or system 22 to securely view health/wellness information from a facility 60 where a patient or resident is being treated in real-time, twenty-four hours a day and seven days a week. As described herein, the facility 60 may refer to a hospital, a nursing facility, a home care facility, a home health facility, a hospice care facility, a palliative care facility, an assisted living facility, a senior housing facility, a residential care facility, an adult day care facility, an ambulance service, a physical therapy facility, a mental health facility, a behavioral health facility, or an addiction rehabilitative facility, among others not explicitly listed herein.

Specifically, the system of FIG. 1 includes a first computing device 4 associated with the first patient/resident guardian 1, a second computing device 5 associated with the second patient/resident guardian 2, and a third computing device 6 associated with a healthcare worker 3 (such as a skilled nursing facility manager, a doctor, a surgeon, a nurse, a manager, a counselor, a psychiatrist, a therapist, a psychologist, a physicians assistant, etc.). It should be appreciated that though two patient/resident guardians are depicted and described herein, a quantity of the patient/resident guardians is not limited to any particular quantity.

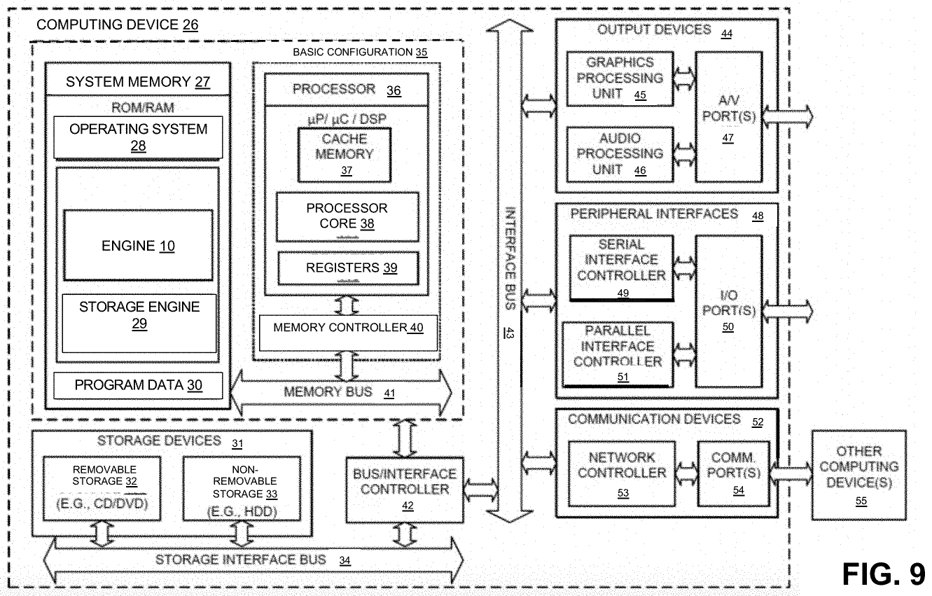

In general, each of the first computing device 4, the second computing device 5, and the third computing device 6 may be a computer, a laptop computer, a smartphone, or a tablet, among other examples not explicitly listed herein. Further, each of the first computing device 4, the second computing device 5, and the third computing device 6 may comprise one or more processors, one or more memories, and one or more computer-readable hardware storage devices, where the one or more computer-readable hardware storage devices contain program code executable by the one or more processors via the one or more memories to implement a method. Such will be described further with reference to FIG. 9 herein.

As shown in FIG. 1, each of the first computing device 4, the second computing device 5, and the third computing device 6 comprise at least an engine 10. In other examples, the engine 10 may be an application, a software program, a service, or a software platform configured to be executable on the first computing device 4, the second computing device 5, and/or the third computing device 6. Further, the system of FIG. 1 may include: a secure storage 8 in the cloud, a secure hosted data store 9, and/or another database (not shown). Each of the secure storage 8 in the cloud, the secure hosted data store 9, and the other database may house information and/or data.

In some instances, each of the secure storage 8 in the cloud, the secure hosted data store 9, and the other database may house profiles, such as a patient profile A 24 (associated with a first patient), a patient profile B 25 (associated with a second patient), a guardian profile A 56 (associated with the first patient/resident guardian 1), a guardian profile B 57 (associated with the second patient/resident guardian 2), a healthcare worker profile A 58 (associated with the healthcare worker 3), etc. It should be appreciated that these profiles are provided for illustrative purposes only. These profiles may include information, such as a name, an address, a job title, a contact number, an emergency contact, an access level to the system 22, historic health/wellness information, current health/wellness information, vital information, etc. As shown, the various components in FIG. 1 are connected via an Internet-based network 7.

In some examples, a graphical user interface (GUI) 21 of the first computing device 4 or the second computing device 5 may receive login credentials from the first patient/resident guardian 1 or the second patient/resident guardian 2. The login credentials may include a username, a password, a biometric identification means (e.g., fingerprint identification, face recognition identification, palm print identification, iris recognition, retina recognition, etc.), etc. In response, the engine 10 may query the secure storage 8 in the cloud, the secure hosted data store 9, and/or the other database to compare the login credentials of the first patient/resident guardian 1 or the second patient/resident guardian 2 with information or data stored in profiles housed in the secure storage 8 in the cloud, the secure hosted data store 9, and/or the other database. In response to the engine 10 detecting a match between the login credentials and a subset of the information in the profile (e.g., the guardian profile A 56 or the guardian profile B 57), the engine 10 is configured to: confirm an identity of the first patient/resident guardian 1 or the second patient/resident guardian 2 from the login credentials and grant/allow the first patient/resident guardian 1 or the second patient/resident guardian 2 access to the system 22. Such access to the system 22 allows the first patient/resident guardian 1 or the second patient/resident guardian 2 to view real-time health/wellness care information about the patient/resident.

Moreover, it should be appreciated that the confirmation of the identity of the first patient/resident guardian 1 or the second patient/resident guardian 2 from the login credentials also includes determining if a given user (e.g., the first patient/resident guardian 1 or the second patient/resident guardian 2) is a “legal guardian” or a person who has been appointed by a court or otherwise has the legal authority to care for the personal and property interests of the patient/resident. If such information is not retrievable from the secure storage 8 in the cloud, the secure hosted data store 9, and/or the other database, the engine 10 may transmit a notification via any messaging means (e.g., email messaging, SMS messaging, etc.) to prompt a third-party user or the facility to confirm or deny such authorization. If such authorization is denied, the first patient/resident guardian 1 or the second patient/resident guardian 2 will be unable to access the system 22.

Similarly, the third computing device 6 may receive the login credentials from the healthcare worker 3. In response, the engine 10 may query the secure storage 8 in the cloud, the secure hosted data store 9, or the other database to compare the login credentials of the healthcare worker 3 with information or data stored in profiles housed in the secure storage 8 in the cloud, the secure hosted data store 9, and/or the other database. In response to the engine 10 detecting a match between the login credentials and a subset of the information in the profile (e.g., the healthcare worker profile A 58), the engine 10 is configured to: confirm an identity of the healthcare worker 3 from the login credentials and grant/allow the healthcare worker 3 access to the system 22.

It should be appreciated that the healthcare worker 3 is given a higher access level to the system 22 as compared to the first patient/resident guardian 1 or the second patient/resident guardian 2. For example, where the first patient/resident guardian 1 or the second patient/resident guardian may be granted authorization to view the health/wellness information of the patient/resident, the healthcare worker 3 may be granted authorization to an administrative interface 23 of the system 22 to add, modify, edit, and delete the health/wellness information of the patient/resident (e.g., input current vitals of the patient/resident).

Further, it should be appreciated that any documentation/file retrieval from external databases is compliant with the Health Insurance Portability and Accountability Act or “HIPAA” governance and data encryption guidelines. Specifically, in accordance with HIPAA guidelines, the system of FIG. 1 will not divulge any protected patient/resident health/wellness information or data in documentation or files unless arrangements have been made to meet HIPAA requirements. In one example, the system of FIG. 1 protects the healthcare records or files of the facility 60 from being forwarded to another file or database by requesting specific authorization for forwarding by the individual registered as the subject, patient or the resident of the file. This extra measure complies with the requirements of the HIPAA. Moreover, in other examples, the system of FIG. 1 may be required to establish an application layer connectivity utilizing Health Level 7 (HL7), Fast Healthcare Interoperability Resources (FHIR), and/or Digital Imaging and Communications in Medicine (DICOM) for imaging prior to searching and retrieving relevant healthcare records.

As described herein, “HL7” refers to a set of international standards for transfer of clinical and administrative data between software applications used by various healthcare providers. As described herein, “FHIR” is a Health Level Seven International® (HL7®) standard for exchanging healthcare information electronically. As described herein, “DICOM” is the standard for the communication and management of medical imaging information and related data.

Once a given user (e.g., the first patient/resident guardian 1, the second patient/resident guardian 2, and/or the healthcare worker 3) is granted access to the system 22, the user may view real-time health/wellness information/data associated with the patient/resident. This health/wellness information includes health/wellness updates regarding the patient/resident of the facility 60. For example, the real-time health/wellness information/data may include: a name of the patient/resident, a photograph of the patient/resident, daily vitals associated with the patient/resident, medications for the patient/resident, meals eaten by the patient/resident, activities engaged in by the patient/resident, etc.

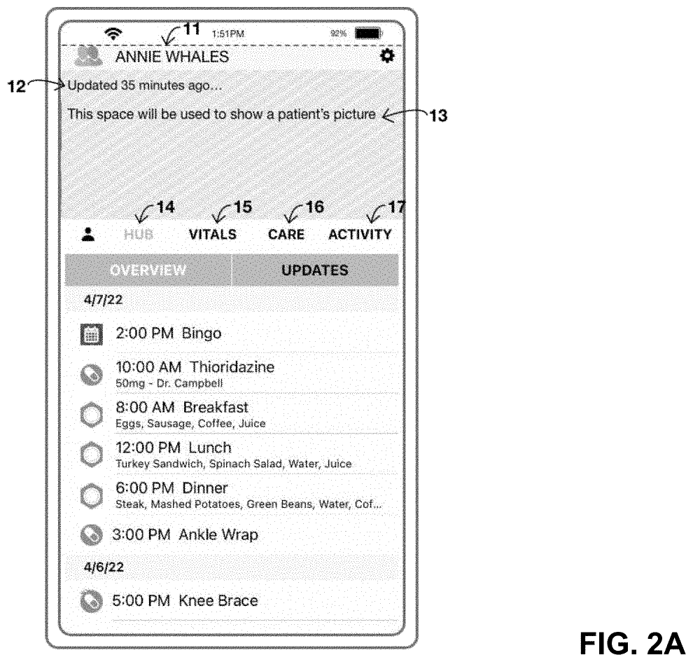

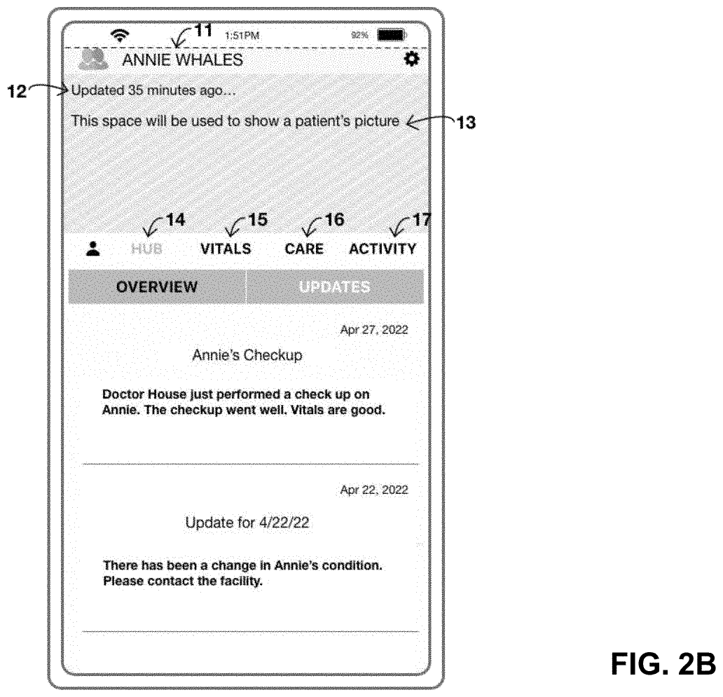

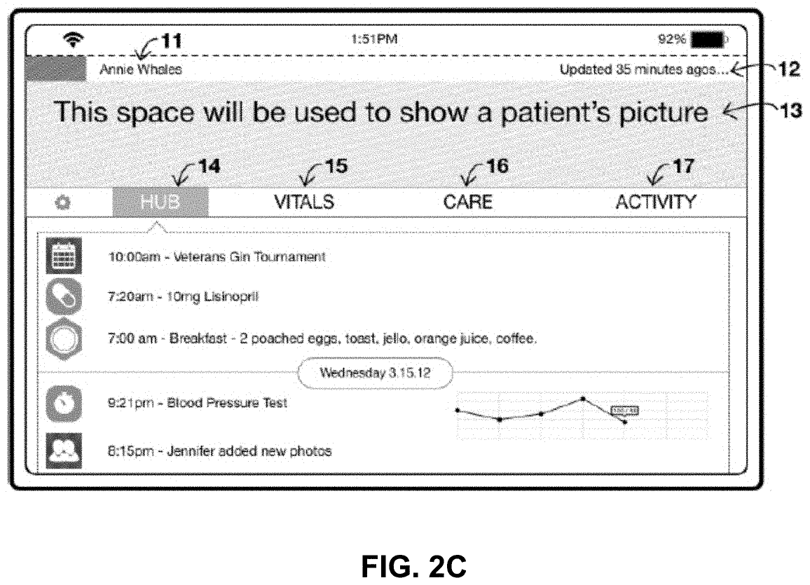

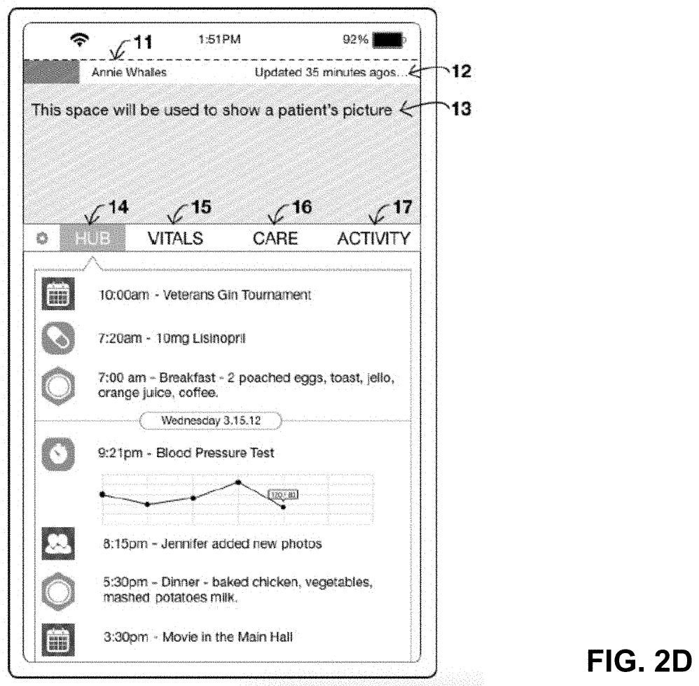

Specifically, FIG. 2A, FIG. 2B, 2C and FIG. 2D illustrate the first patient/resident guardians 1 or the second patient/resident guardians 2 views of a patients/residents hub/dashboard 14 of the system 22. As shown in FIG. 2A, FIG. 2B, 2C and FIG. 2D, these views may include a name 11 of the patient/resident, a timeframe 12 when the patients/residents hub/dashboard was updated last, an image 13 of the patient/resident, an overview of all activities engaged in by the patient/resident on a particular day and the times when these activities were completed, real-time direct wellness updates sent directly from facility 60 (as shown in FIG. 2B), etc.

This information may be manually input into the system 22 by the healthcare worker 3 or may be captured from healthcare records or documentation via a data extraction method (e.g., via optical character recognition or OCR). For example and as shown in FIG. 2A, this view shows that on Apr. 7, 2022, patient/resident Annie Whales was given 50 mg of the medication Thioridazine from Dr. Campbell at 10:00 AM and lunch (e.g., a turkey sandwich and a spinach salad) at 12:00 PM. In some examples, this view may also depict medications of the patient/resident.

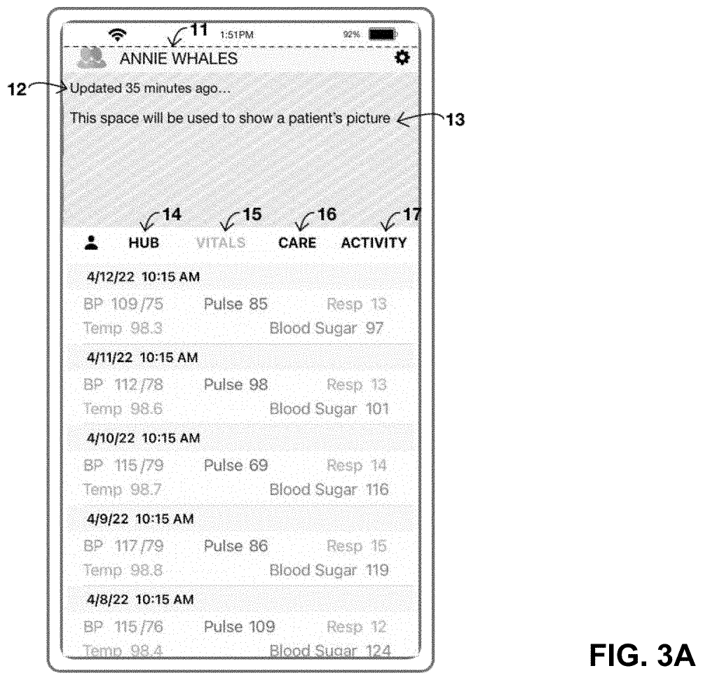

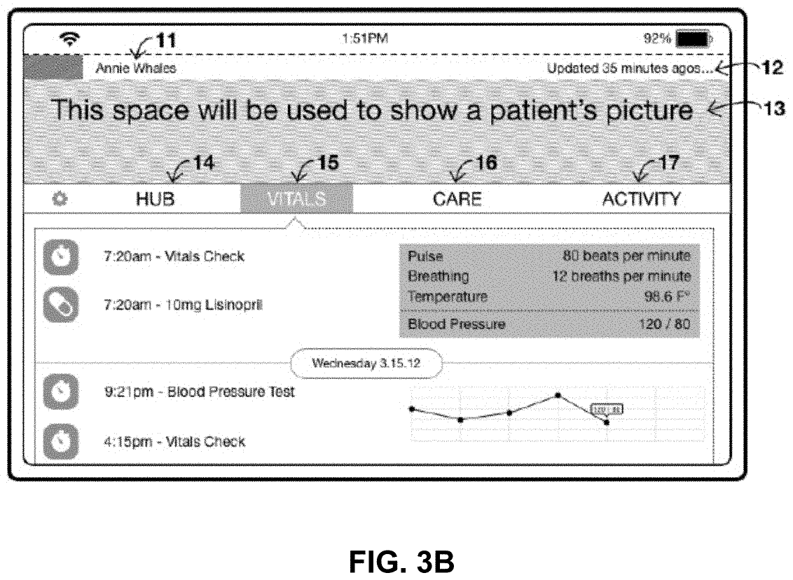

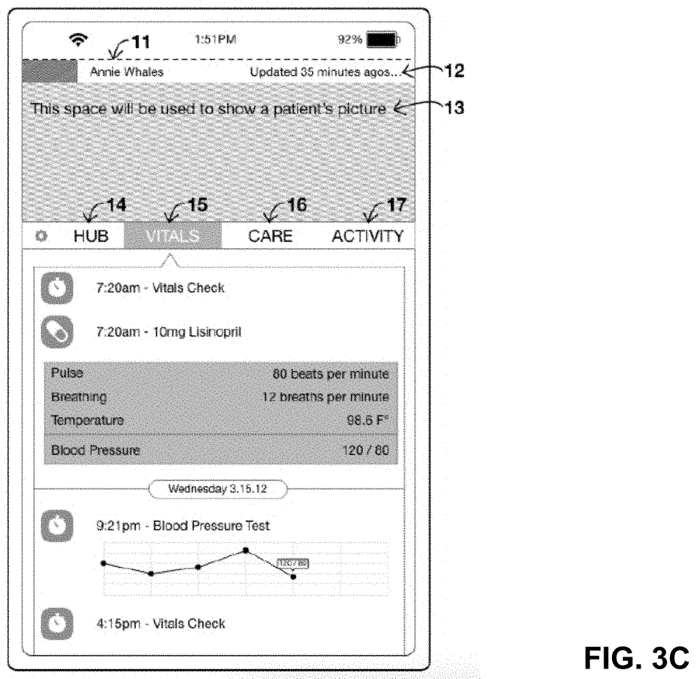

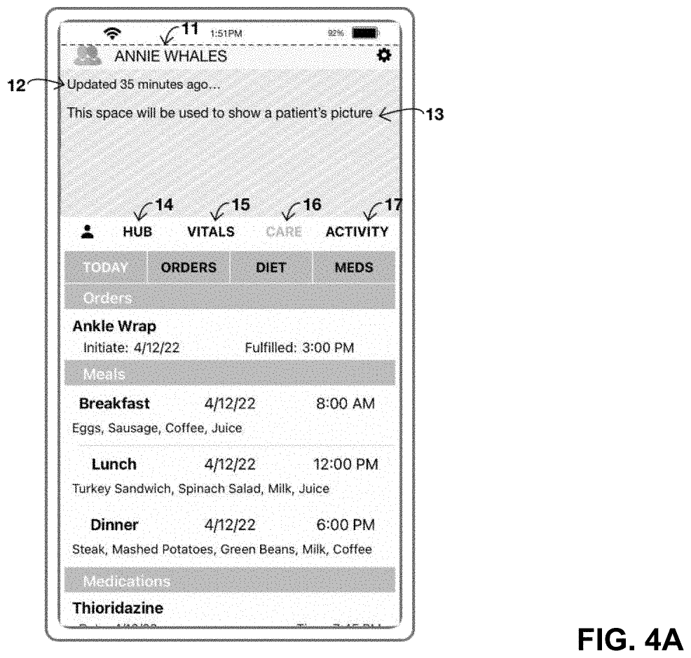

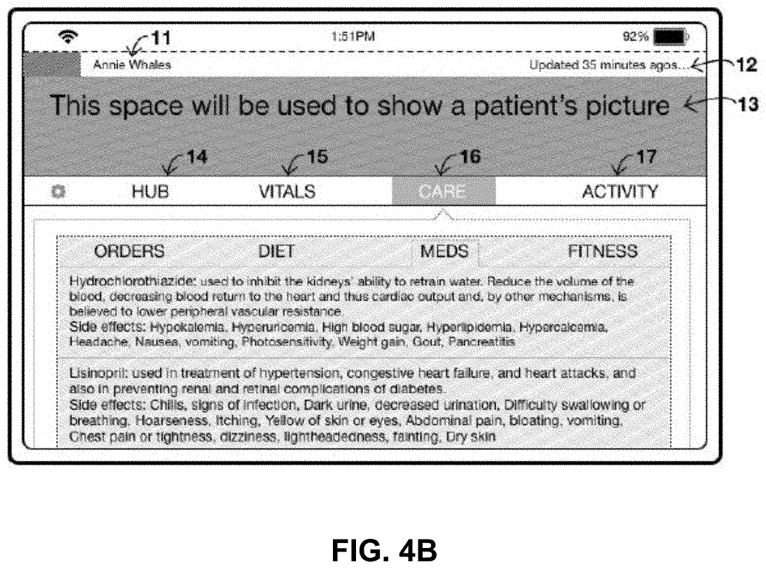

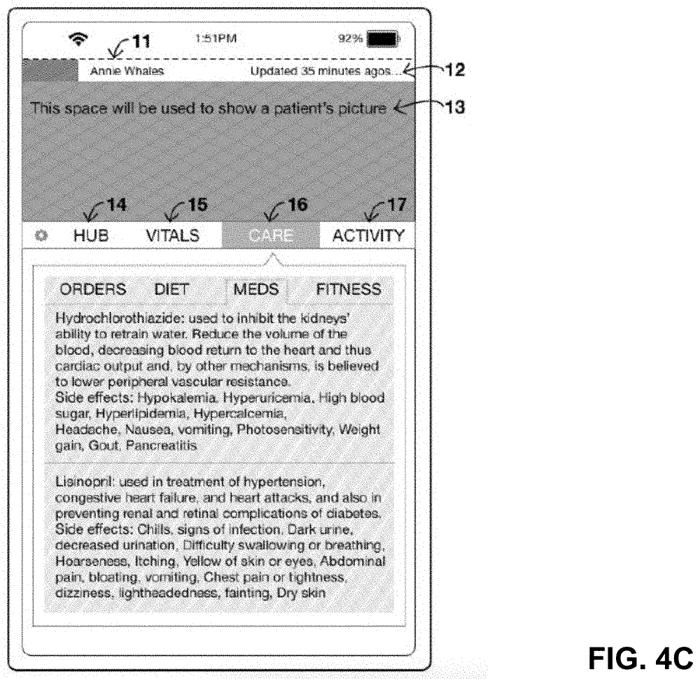

FIG. 3A, FIG. 3B, and FIG. 3C illustrate the first patient/resident guardians 1 or the second patient/resident guardians 2 views of the vitals 15 of the patient/resident. These real-time vitals 15 may include, but are not limited to, blood pressure, pulse rate, body temperature, blood sugar level, respiration rate, etc. FIG. 4A, FIG. 4B, and FIG. 4C illustrate the first patient/resident guardians 1 or the second patient/resident guardians 2 views of patient/resident care information 16, which may include the most up-to-date information on the patients/residents care, such as: recent doctor's orders (e.g., Annie Whales ankle was wrapped on Apr. 12, 2022 at 3:00 PM), diet restrictions, prescribed medications with brief usage and side effects, among others not explicitly listed herein. This information of FIG. 3A-FIG. 4C may be manually input into the system 22 by the healthcare worker 3 or may be captured from healthcare records or documentation via a data extraction method (e.g., via OCR).

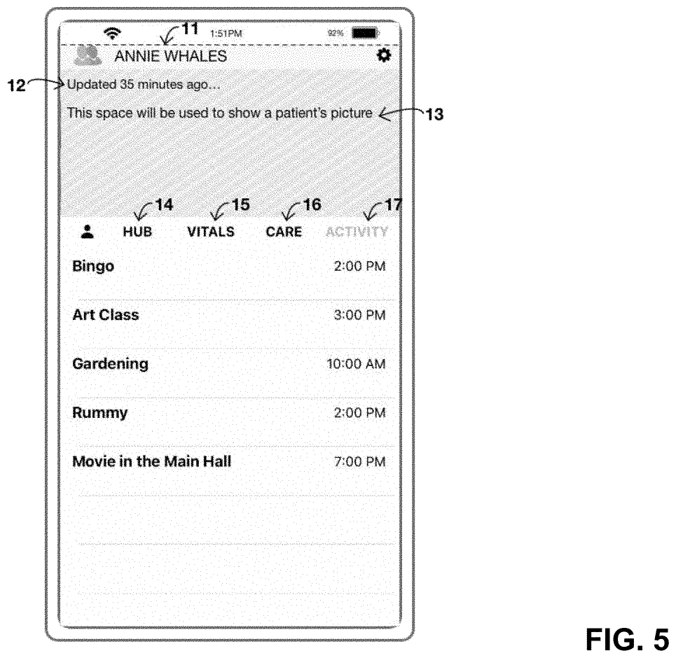

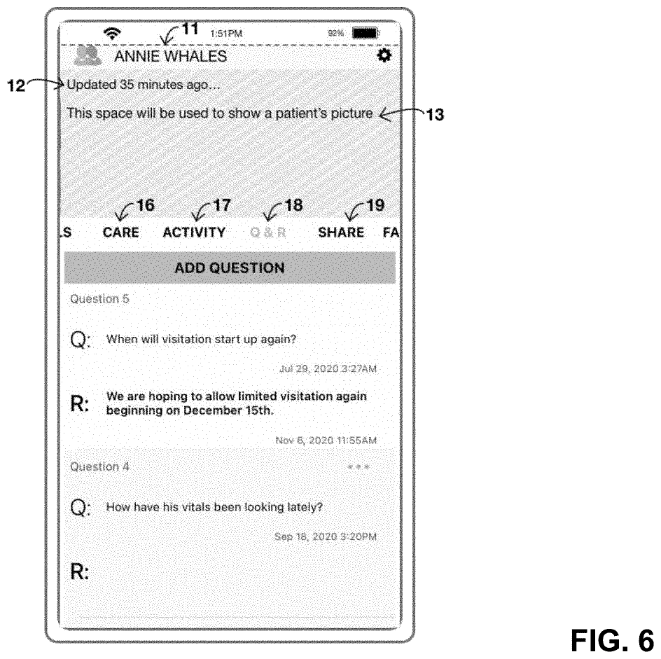

Further, FIG. 5 illustrates the first patient/resident guardians 1 or the second patient/resident guardians 2 view of patient/resident activity information 17, which may include activities engaged in on a daily basis for the patient/resident (e.g., bingo, art class, gardening, etc.), as well as the time of the day that these activities were engaged in. This information of FIG. 5 may be manually input into the system 22 by the healthcare worker 3. FIG. 6 illustrates the first patient/resident guardians 1 or the second patient/resident guardians 2 view of a question and response (Q & R) feature/component 18 of the system 22. The Q & R feature/component 18 allows the first patient/resident guardian 1 or the second patient/resident guardian 2 to ask questions and receive responses from the healthcare worker 3 or another authorized personnel at the facility 60 in real-time.

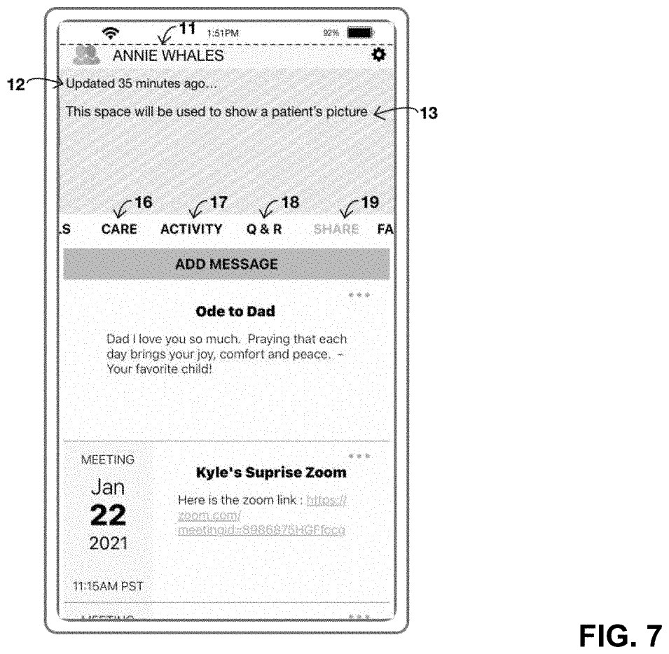

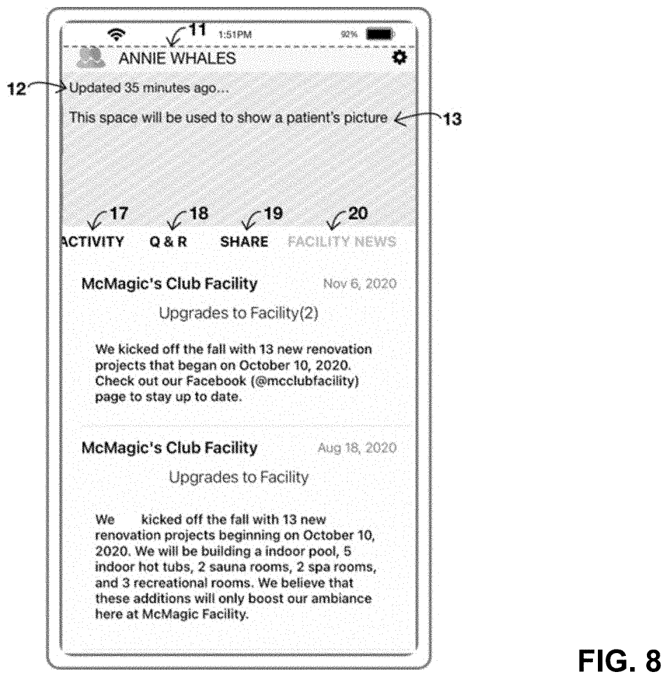

FIG. 7 illustrates the first patient/resident guardians 1 or the second patient/resident guardians 2 view of a share feature/component 19 of the system 22. This feature allows the first patient/resident guardian 1 or the second patient/resident guardian 2 to transmit meetings or event information via SMS or email messaging to a third-party. FIG. 8 illustrates the first patient/resident guardians 1 or the second patient/resident guardians 2 view of a facility news feature/component 20 of the system 22, which depicts the most up-to-date information or news regarding the facility 60.

Thus, as described herein, the present invention allows the first patient/resident guardian 1 and/or the second patient/resident guardian 2 to access the system 22 to securely view and retrieve health/wellness information associated with the patient/resident of the facility 60. It should be appreciated that there are no current process available that provide guardians with this real-time patient health/wellness care updates in a secure manageable method.

### Computing System

FIG. 9 is a block diagram of a computing device included within the computer system of FIG. 1. A basic configuration 35 of a computing device 26 is illustrated in FIG. 9 by those components within the inner dashed line. In the basic configuration 35 of the computing device 26, the computing device 26 includes a processor 36 and a system memory 27. In some examples, the computing device 26 may include one or more processors and the system memory 27. A memory bus 41 is used for communicating between the one or more processors 36 and the system memory 27.

Depending on the desired configuration, the processor 36 may be of any type, including, but not limited to, a microprocessor (μP), a microcontroller (μC), and a digital signal processor (DSP), or any combination thereof. Further, the processor 36 may include one more levels of caching, such as a level cache memory 37, a processor core 38, and registers 39, among other examples. The processor core 38 may include an arithmetic logic unit (ALU), a floating point unit (FPU), and/or a digital signal processing core (DSP Core), or any combination thereof. A memory controller 40 may be used with the processor 36.

Depending on the desired configuration, the system memory 27 may be of any type, including, but not limited to, volatile memory (such as RAM), and/or non-volatile memory (such as ROM, flash memory, etc.), or any combination thereof. The system memory 27 includes an operating system 28, one or more engines, such as the engine 10, and program data 30. The system memory 27 may also include a storage engine 29 that may store any information or data disclosed herein.

Moreover, the computing device 26 may have additional features or functionality, and additional interfaces to facilitate communications between the basic configuration 35 and any desired devices and interfaces. For example, a bus/interface controller 42 is used to facilitate communications between the basic configuration 35 and data storage devices 31 via a storage interface bus 234. The data storage devices 31 may be one or more removable storage devices 32, one or more non-removable storage devices 33, or a combination thereof. Examples of the one or more removable storage devices 32 and the one or more non-removable storage devices 33 include magnetic disk devices (such as flexible disk drives and hard-disk drives (HDD)), optical disk drives (such as compact disk (CD) drives or digital versatile disk (DVD) drives), solid state drives (SSD), and tape drives, among others.

In some embodiments, an interface bus 43 facilitates communication from various interface devices (e.g., one or more output devices 44, one or more peripheral interfaces 48, and one or more communication devices 52) to the basic configuration 35 via the bus/interface controller 42. Some of the one or more output devices 44 include a graphics processing unit 45 and an audio processing unit 46, which are configured to communicate to various external devices, such as a display or speakers, via one or more A/V ports 47.

The one or more peripheral interfaces 48 may include a serial interface controller 49 or a parallel interface controller 51, which are configured to communicate with external devices, such as input devices (e.g., a keyboard, a mouse, a pen, a voice input device, or a touch input device, etc.) or other peripheral devices (e.g., a printer or a scanner, etc.) via one or more I/O ports 50.

Further, the one or more communication devices 52 may include a network controller 53, which is arranged to facilitate communication with one or more other computing devices 55 over a network communication link via one or more communication ports 54. The one or more other computing devices 55 include servers, the database, mobile devices, and comparable devices.

The network communication link is an example of a communication media. The communication media are typically embodied by the computer-readable instructions, data structures, program modules, or other data in a modulated data signal, such as a carrier wave or other transport mechanism, and include any information delivery media. A “modulated data signal” is a signal that has one or more of its characteristics set or changed in such a manner as to encode information in the signal. By way of example, and not limitation, the communication media may include wired media (such as a wired network or direct-wired connection) and wireless media (such as acoustic, radio frequency (RF), microwave, infrared (IR), and other wireless media). The term “computer-readable media,” as used herein, includes both storage media and communication media.

It should be appreciated that the system memory 27, the one or more removable storage devices 32, and the one or more non-removable storage devices 33 are examples of the computer-readable storage media. The computer-readable storage media is a tangible device that can retain and store instructions (e.g., program code) for use by an instruction execution device (e.g., the computing device 26). Any such, computer storage media is part of the computing device 26.

The computer readable storage media/medium can be a tangible device that can retain and store instructions for use by an instruction execution device. The computer readable storage media/medium may be, for example, but is not limited to, an electronic storage device, a magnetic storage device, an optical storage device, an electromagnetic storage device, and/or a semiconductor storage device, or any suitable combination of the foregoing. A non-exhaustive list of more specific examples of the computer readable storage media/medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, and/or a mechanically encoded device (such as punch-cards or raised structures in a groove having instructions recorded thereon), and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Aspects of the present invention are described herein regarding illustrations and/or block diagrams of methods, computer systems, and computing devices according to embodiments of the invention. It will be understood that each block in the block diagrams, and combinations of the blocks, can be implemented by the computer-readable instructions (e.g., the program code).

The computer-readable instructions are provided to the processor 36 of a general purpose computer, special purpose computer, or other programmable data processing apparatus (e.g., the computing device 25) to produce a machine, such that the instructions, which execute via the processor 36 of the computer or other programmable data processing apparatus, create means for implementing the functions/acts specified in the block diagram blocks. These computer-readable instructions are also stored in a computer-readable storage medium that can direct a computer, a programmable data processing apparatus, and/or other devices to function in a particular manner, such that the computer-readable storage medium having instructions stored therein comprises an article of manufacture including instructions, which implement aspects of the functions/acts specified in the block diagram blocks.

The computer-readable instructions (e.g., the program code) are also loaded onto a computer (e.g. the computing device 26), another programmable data processing apparatus, or another device to cause a series of operational steps to be performed on the computer, the other programmable apparatus, or the other device to produce a computer implemented process, such that the instructions, which execute on the computer, the other programmable apparatus, or the other device, implement the functions/acts specified in the block diagram blocks.

Computer readable program instructions described herein can also be downloaded to respective computing/processing devices from a computer readable storage medium or to an external computer or external storage device via a network (e.g., the Internet, a local area network, a wide area network, and/or a wireless network). The network may comprise copper transmission cables, optical transmission fibers, wireless transmission, routers, firewalls, switches, gateway computers, and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer/computing device, partly on the user's computer/computing device, as a stand-alone software package, partly on the user's computer/computing device and partly on a remote computer/computing device or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to block diagrams of methods, computer systems, and computing devices according to embodiments of the invention. It will be understood that each block and combinations of blocks in the diagrams, can be implemented by the computer readable program instructions.

The block diagrams in the Figures illustrate the architecture, functionality, and operation of possible implementations of computer systems, methods, and computing devices according to various embodiments of the present invention. In this regard, each block in the block diagrams may represent a module, a segment, or a portion of executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block and combinations of blocks can be implemented by special purpose hardware-based systems that perform the specified functions or acts or carry out combinations of special purpose hardware and computer instructions.

Another embodiment of the invention provides a method that performs the process steps on a subscription, advertising, and/or fee basis. That is, a service provider can offer to assist in the method steps described herein. In this case, the service provider can create, maintain, and/or support, etc. a computer infrastructure that performs the process steps for one or more customers. In return, the service provider can receive payment from the customer(s) under a subscription and/or fee agreement, and/or the service provider can receive payment from the sale of advertising content to one or more third parties.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others or ordinary skill in the art to understand the embodiments disclosed herein.

When introducing elements of the present disclosure or the embodiments thereof, the articles “a,” “an,” and “the” are intended to mean that there are one or more of the elements. Similarly, the adjective “another,” when used to introduce an element, is intended to mean one or more elements. The terms “including” and “having” are intended to be inclusive such that there may be additional elements other than the listed elements.

Although this invention has been described with a certain degree of particularity, it is to be understood that the present disclosure has been made only by way of illustration and that numerous changes in the details of construction and arrangement of parts may be resorted to without departing from the spirit and the scope of the invention.

What is claimed is:

1. A system comprising:
a first computing device associated with a guardian of a patient or a resident at a facility;
a second computing device associated with a healthcare worker of the facility, wherein each of the first computing device and the second computing device comprise at least an engine and a graphical user interface (GUI);
at least one database configured to store a first profile associated with the patient or the resident and a second profile associated with the guardian, wherein the first profile houses medical documentation or records associated with the patient or the resident; and
a network configured to connect the first computing device, the second computing device, and the at least one database,
wherein the engine of the first computing device is configured to:
receive login credentials via the GUI from the guardian;
query the at least one database to compare the login credentials with information stored in the second profile; and
in response to identifying a match between the login credentials and a subset of the information in the second profile,
confirming an identity of the guardian; and
granting the guardian access to view real-time health or wellness information associated with the patient or the resident.

2. The system of claim 1, wherein the facility is selected from the group consisting of: a hospital, a nursing facility, a home care facility, a home health facility, a hospice care facility, a palliative care facility, an assisted living facility, a senior housing facility, a residential care facility, an adult day care facility, an ambulance service facility, a physical therapy facility, a mental health facility, a behavioral health facility, and an addiction rehabilitative facility.

3. The system of claim 1, wherein the healthcare worker is selected from the group consisting of: a doctor, a surgeon, a nurse, a manager, a counselor, a psychiatrist, a therapist, a psychologist, and a physicians assistant.

4. The system of claim 1, wherein the login credentials are selected from the group consisting of: a username, a password, and a biometric identification means.

5. The system of claim 4, wherein the biometric identification means are selected from the group consisting of: fingerprint identification, face recognition identification, palm print identification, iris recognition, and retina recognition.

6. The system of claim 1, wherein the engine of the first computing device is further configured to:
determine, from the login credentials, if the guardian has a legal authorization to view the wellness care updates associated with the patient or the resident.

7. The system of claim 6, wherein, in response to a failure to identify the guardian as having the legal authorization to view the wellness care updates associated with the patient or the resident, the engine is further configured to:
transmit a notification to a third-party user or the facility to confirm or deny the authorization; and
prohibit the guardian from viewing the wellness care updates associated with the patient or the resident.

8. The system of claim 1, wherein the guardian is associated with a first access level associated with the wellness care updates, and wherein the healthcare worker of the facility is associated with a second access level associated with the wellness care updates.

9. The system of claim 8, wherein the second access level is greater than the first access level.

10. The system of claim 1, wherein retrieval of the medical documentation or the records from the at least one database is compliant with Health Insurance Portability and Accountability Act (HIPAA) governance.

11. The system of claim 1, wherein the real-time health or wellness information associated with the patient or the resident is selected from the group consisting of: vitals associated with the patient or the resident, medications of the patient or the resident, meals eaten by the patient or the resident, activities engaged in by the patient or the resident, doctors' orders or instructions for the patient or the resident, and dietary restrictions for the patient or the resident.

12. The system of claim 11, wherein the vitals associated with the patient or the resident are selected from the group consisting of: blood pressure, pulse rate, body temperature, blood sugar level, and respiration rate.

13. The system of claim 1, wherein the engine comprises a question and response (Q & R) component that allows the guardian to ask a question and receive an answer from the healthcare worker of the facility.

14. The system of claim 1, wherein the engine comprises a share component that allows the guardian to share meetings or textual information to a third-party.

15. The system of claim 1, wherein the engine comprises a facility news component that provides the guardian with real-time information regarding the facility.
